(19) United States
(12) Patent Application Publication
(10) Pub. No.: US 2010/0144801 A1
(43) Pub. Date: Jun. 10, 2010

(54) MUSCARINIC RECEPTOR ANTAGONISTS

(76) Inventors: Naresh Kumar, Gurgaon (IN); Jaskiran Kaur, Amritsar (IN); Abhijit Ray, New Delhi (IN); Suman Gupta, Gurgaon (IN); Shivani Malhotra, New Delhi (IN); Rajkumar Shirumalla, New Delhi (IN)

Correspondence Address: Ranbaxy Inc., Intellectual Property Department, 600 College Road East, PRINCETON, NJ, 08540, US

(21) Appl. No.: 12/443,838

(22) PCT Filed: Oct. 2, 2007
(86) PCT No.: PCT/IB2007/054008

§ 371 Date: Dec. 23, 2009

(30) Foreign Application Priority Data
Oct. 3, 2006 (IN) 2178/DEL/2006

(51) Int. Cl. A61K 31/44; C07D 213/55; C07D 233/54; A61K 31/4164; A61P 11/00; A61P 13/00; A61P 1/00; A61P 3/00

(52) U.S. Cl. 514/357; 546/335; 548/338.1; 514/400

(57) ABSTRACT

The present invention generally relates to muscarinic receptor antagonists of formula I, which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors. Formula (I) wherein Het: is heterocyclyl or heteroaryl X: O, S or NR1 and the other substituents are defined as in the claims.

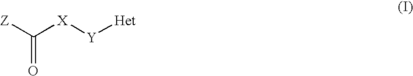

### FIELD OF THE INVENTION

This present invention generally relates to muscarinic receptor antagonists which are useful, among other uses, for the treatment of various diseases of the respiratory, urinary and gastrointestinal systems mediated through muscarinic receptors. The present invention also relates to the process for the preparation of disclosed compounds, pharmaceutical compositions containing the disclosed compounds, and the methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the super family of G-protein coupled receptors and five molecularly distinct subtypes are known to exist (M₁, M₂, M₃, M₄ and Mₛ).

These receptors are widely distributed on multiple organs and tissues and are critical to the maintenance of central and peripheral cholinergic neurotransmission. The regional distribution of these receptor sub-types in the brain and other organs has been documented (for example, the M₁ subtype is located primarily in neuronal tissues, such as cerebral cortex and autonomic ganglia, the M₂ subtype is present mainly in the heart and bladder smooth muscle, and the M₃ subtype is located predominantly on smooth muscle and salivary glands (Nature, 323:411 (1986); Science, 237:527 (1987)).

A review in Curr. Opin. Chem. Biol., 3:426 (1999), as well as in Trends in Pharmacal. Sci., 22:409 (2001) by Eglen et al., describes the biological potentials of modulating muscarinic receptor subtypes by ligands in different disease conditions, such as Alzheimer's disease, pain, urinary disease condition, chronic obstructive pulmonary disease, and the like.

The pharmacological and medical aspects of the muscarinic class of acetylcholine agonists and antagonists are presented in a review in Molecules, 6:142 (2001). Birdsall et al. Trends in Pharmacal. Sci., 22:215 (2001) has also summarized the recent developments on the role of different muscarinic receptor subtypes using different muscarinic receptor of knock out mice.

Almost all smooth muscle express a mixed population of M₂ and M₃ receptors. Although M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However, the therapeutic utility of antimuscarinics has been limited by poor tolerability as a result of treatment related, frequent systemic adverse events, such as dry mouth, constipation, blurred vision, headache, somnolence and tachycardia. Thus, there exists a need for novel muscarinic receptor antagonists that demonstrate target organ selectivity.

WO 2004/005252 discloses azabicyclo derivatives described as muscarinic receptor antagonists. WO 2004/004629, WO 2004/052857, WO 2004/067510, WO 2004/014853 and WO 2004/014363 disclose 3,6-disubstituted azabicyclo [3.1.0] hexane derivatives described as useful muscarinic receptor antagonists. WO 2004/056811 discloses flaxavate derivatives as muscarinic receptor antagonists. WO 2004/056810 discloses xanthene derivatives as muscarinic receptor antagonists. WO 2004/056767 discloses 1-substituted-3-pyrrolidine derivatives as muscarinic receptor antagonists. WO 99/14200, WO 03/027060, U.S. Pat. No. 6,200,991 and WO 00/56718 disclose heterocycle derivatives as muscarinic receptor antagonists. WO 2004/089363, WO 2004/089898, WO 2004/069835, WO 2004/089900 and WO 2004/089364 disclose substituted azabicyclohexane derivatives as muscarinic receptor antagonists. WO2005/026121 discloses process for the preparation of azabicyclohexane derivatives. WO2006/018708 discloses pyrrolidine derivatives as muscarinic receptor antagonists. WO2006/054162, WO2006/016245, WO2006/016345, WO2006/05282 and WO2006/35303 disclose azabicyclo derivatives as muscarinic receptor antagonists. WO2006/032994 discloses amine derivatives as muscarinic receptor antagonists.

J. Med. Chem., 44:984 (2002), describes cyclohexylmethylpiperidinyl-triphenylpropioamide derivatives as selective M₃ antagonist discriminating against the other receptor subtypes. J. Med. Chem., 36:610 (1993), describes the synthesis and antimuscarinic activity of some 1-cycloalkyl-1-hydroxy-1-phenyl-3-(4-substituted piperazinyl)-2-propanones and related compounds. J. Med. Chem., 34:3065 (1991), describes analogues of oxybutynin, synthesis and antimuscarinic activity of some substituted 7-amino-1-hydroxy-5-heptyn-2-ones and related compounds. Bio-Organic Medicinal Chemistry Letters, 15:2093 (2005) describes synthesis and activity of analogues of oxybutynin and tolterodine. Chem. Pharma. Bull. 53(4):437, 2005 discloses thiazole carboxamide derivatives.

However, there remains a need for novel muscarinic receptor antagonists useful in treating disease states associated with improper smooth muscle function and respiratory disorders.

### SUMMARY OF THE INVENTION

Accordingly, provided herein are novel compounds that can be useful in treating disease states associated with improper smooth muscle function and respiratory disorders.

Thus, in one aspect, there are provided compounds having the structure of Formula I:

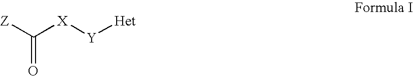

or a pharmaceutically accepted salts, pharmaceutically acceptable solvates, enantiomers, diastereomers, polymorphs or N-oxides
Het is heterocyclyl or heteroaryl wherein nitrogen atom in heterocyclyl ring may also be quaternized to form a quaternary ammonium salts,
X is O, S or —NR₁ (wherein R₁ is as defined below);
Y is no atom or —(CH₂)ₙ;
n is an integer from 1 to 6;
Z is —NHR₂, —N(R₂)₂ (wherein R₂ is as defined below), aryl or cycloalkyl;
R₁ is hydrogen, alkyl or aralkyl;
R₂ is independently selected from alkyl, aryl, aralkyl, heteroaryl, cycloalkyl, heterocyclyl, heterocyclylalkyl or heteroarylalkyl;

In another aspect, provided are pharmaceutical compositions comprising a therapeutically effective amount of a compound described herein and one or more pharmaceutically acceptable carriers, excipients or diluents. The pharmaceutical compositions can further comprise one or more corticosteroids, beta agonists, leukotriene antagonists, 5-lipoxygenase inhibitors, anti-histamines, antitussives, dopamine receptor antagonists, chemokine inhibitors, p38 MAP Kinase inhibitors, or PDE-IV inhibitors or a mixture thereof.

In another aspect, provided are methods of treating or preventing disease or disorder of the respiratory, urinary or gastrointestinal systems, wherein the disease or disorder is mediated through muscarinic receptors, comprising administering to a patient in need thereof a therapeutically effective amount of a compound described herein.

In yet another aspect, provided are methods of treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis comprising administering to a patient in need thereof a therapeutically effective amount of a compound described herein.

In another aspect, provided are methods of preparing a compound of Formula VII comprising the steps of:
reacting a compound of Formula II

(R₂)₂NH Formula II
with a compound of Formula III

hal-COOR_{z} Formula III
to form a compound of Formula IV,

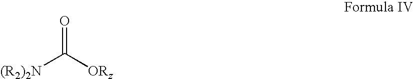

reacting a compound of Formula IV with a compound of Formula V

het-Y—OH Formula V
to give a compound of Formula VI

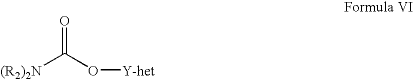

reducing a compound of Formula VI (when het is pyridyl) followed by reaction with a compound of Formula P-hal to give a compound of Formula VII

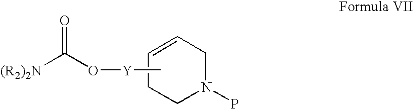

wherein
Het is heterocyclyl or heteroaryl wherein nitrogen atom in heterocyclyl ring may also be quaternized to form a quaternary ammonium salts,
Y is no atom or —(CH₂)ₙ;
R₂ is independently selected from alkyl, aryl, aralkyl, heteroaryl, cycloalkyl, heterocyclyl, heterocyclylalkyl or heteroarylalkyl;
R_{z} is alkyl or aryl;
hal Cl, Br, I;
P is selected from alkyl, cycloalkyl, heteroaryl, heterocyclyl, heteroarylalkyl, heterocyclylalkyl, aralkyl, —C(═O)OC(CH₃)₃, —C(═O)OC(CH₃)₂CHBr₂ or —C(═O)OC(CH₃)₂CCl₃;

### DETAILED DESCRIPTION OF THE INVENTION

In one aspect, there are provided muscarinic receptor antagonist, which can be useful and effective therapeutic or prophylactic agents for the treatment of various diseases of the respiratory, urinary and gastrointestinal systems. Also provided are processes for synthesizing such compounds.

In another aspects, pharmaceutical composition containing such compounds are provided together with one or more pharmaceutically acceptable carriers, excipients or diluents, which can be useful for the treatment of various diseases of the respiratory, urinary and gastrointestinal systems.

Enantiomers, diastereomers, N-oxides, polymorphs, pharmaceutically acceptable salts and pharmaceutically acceptable solvates of compounds described herein, as well as metabolites having the same type of activity, are also provided, as well as pharmaceutical compositions thereof in combination with one or more pharmaceutically acceptable carriers, excipients or dilutents.

Other aspects will be set forth in the description which follows, and in part will be apparent from the description or may be learnt by the practice of the invention.

Thus in one aspect, there are provided compounds having the structure of Formula I:

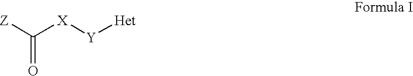

or a pharmaceutically accepted salts, pharmaceutically acceptable solvates, enantiomers, diastereomers, polymorphs or N-oxides
Het is heterocyclyl or heteroaryl wherein nitrogen atom in heterocyclyl ring may also be quaternized to form a quaternary ammonium salts,
X is O, S or —NR₁ (wherein R₁ is as defined below);
Y is no atom or —(CH₂)ₙ;
n is an integer from 1 to 6;
Z is —NHR₂, —N(R₂)₂ (wherein R₂ is as defined below), aryl or cycloalkyl;
R₁ is hydrogen, alkyl or aralkyl;
R₂ is independently selected from alkyl, aryl, aralkyl, heteroaryl, cycloalkyl, heterocyclyl, heterocyclylalkyl or heteroarylalkyl;

The following definitions apply to terms as used herein:

The term “alkyl” unless otherwise specified refers to a monoradical branched or unbranched saturated hydrocarbon chain having from 1 to 20 carbon atoms. Groups such as methyl, ethyl, n-propyl, iso-propyl, n-butyl, iso-butyl, t-butyl, n-hexyl, n-decyl, tetradecyl, and the like exemplify this term. Alkyl may further be substituted with one or more substituents selected from the group consisting of alkenyl, alkynyl, hydroxyl, alkoxy, aryloxy, cycloalkyl, acyl, acylamino, acyloxy, —NHC(═O)OR₂, azido, cyano, halogen, thiocarbonyl, substituted thiocarbonyl, carboxy, —COOR₂ (wherein R₂ is as defined earlier), thiol, alkoxyamino, —NRₓR_{y} (Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also together join to form a heterocyclyl ring), —C(═O)NRₓR_{y}, —OC(═O)NRₓR_{y}, —NHC(═O)NRₓR_{y} (wherein Rₓ and R_{y} are the same as defined earlier), nitro, —S(O)ₙR₃ (wherein R₃ is alkyl, aryl, heteroaryl, heterocyclyl, cycloalkyl, aralkyl, heteroarylalkyl, heterocyclylalkyl or —NRₓR_{y} (where Rₓ and R_{y} are the same as defined earlier) and n is 0, 1 or 2). Unless otherwise constrained by the definition, all substituents may be further substituted by 1-3 substituents chosen from alkyl, carboxy, —COOR₂ (wherein R₂ is the same as defined earlier), —NRₓR_{y}, —C(═O)NRₓR_{y}, —NHC(═O)NRₓR_{y} (wherein Rₓ and R_{y} are the same as defined earlier), —NHC(═O)OR₂, hydroxy, alkoxy, halogen, —CF₃, cyano, and —S(O)ₙR₃ (where n and R₃ are the same as defined earlier). Alkyl group as defined above may also be interrupted by 1-5 atoms of groups independently chosen from oxygen, sulfur and —NRₐ (where Rₐ is chosen from hydrogen, alkyl, cycloalkyl, aryl).

The term “alkenyl” unless otherwise specified refers to a monoradical of a branched or unbranched unsaturated hydrocarbon group preferably having 2 to 20 carbon atoms with cis or trans geometry. Preferred alkenyl groups include ethenyl or vinyl, 1-propylene or allyl, iso-propylene, bicyclo[2.2.1]heptene, and the like. In the event that alkenyl is attached to the heteroatom, the double bond cannot be alpha to the heteroatom. It may further be substituted with one or more substituents selected from the group consisting of alkyl, alkynyl, alkoxy, acyl, acylamino, acyloxy, —CF₃, —NRₓR_{y}, —C(═O)NRₓR_{y}, —OC(═O)NRₓR_{y}, —NHC(═O)NRₓR_{y} (wherein Rₓ and R_{y} are the same as defined earlier), —NHC(═O)ORₓ, azido, cyano, halogen, hydroxy, thiocarbonyl, substituted thiocarbonyl, carboxy, —COOR₂ (wherein R₂ is the same as defined earlier), thiol, aryl, aralkyl, aryloxy, cycloalkyl, heterocyclyl, heteroaryl, heterocyclylalkyl, heteroarylalkyl, —NHORₓ, nitro, S(O)ₙR₃ (wherein n and R₃ are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, —CF₃, cyano, —NRₓR_{y}, —C(═O)NRₓR_{y}, —OC(═O)NRₓR_{y} (wherein Rₓ and R_{y} are the same as defined earlier) and —S(O)ₙR₃ (where R₃ and n are the same as defined earlier).

The term “alkynyl” unless otherwise specified refers to a monoradical of an unsaturated hydrocarbon, preferably having from 2 to 20 carbon atoms. Preferred alkynyl groups include ethynyl, propargyl or propynyl, and the like. In the event that alkynyl is attached to the heteroatom, the triple bond cannot be alpha to the heteroatom. It may further be substituted with one or more substituents selected from the group consisting of alkyl, alkenyl, alkoxy, cycloalkyl, acyl, acylamino, alkoxyamino, acyloxy, alkoxycarbonylamino, azido, cyano, halogen, hydroxy, thiocarbonyl, substituted thiocarbonyl, —CF₃, carboxy, —COOR₂ (wherein R₂ is the same as defined earlier), thiol, aryl, aralkyl, aryloxy, nitro, heterocyclyl, heteroaryl, heterocyclylalkyl, heteroarylalkyl, —NRₓR_{y}, —C(═O)NRₓR_{y}, —OC(═O)NRₓR_{y}, —NHC(═O)NRₓR_{y} (wherein Rₓ and R_{y} are the same as defined earlier), —S(O)ₙR₃ (wherein n and R₃ are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, —CF₃, —C(═O)NRₓR_{y}, —OC(═O)NRₓR_{y} (wherein Rₓ and R_{y} are the same as defined earlier), cyano and —S(O)ₙR₃ (wherein R₃ and n are the same as defined earlier).

The term “alkylene,” unless otherwise specified, refers to a diradical branched or unbranched saturated hydrocarbon chain having from 1 to 6 carbon atoms. This term can be exemplified by groups such as methylene, ethylene, propylene isomers and the like. Alkylene may further be substituted with one or more substituents selected from alkyl, alkenyl, alkynyl, alkoxy, cycloalkyl, acyl, acylamino, acyloxy, alkoxycarbonylamino, azido, cyano, halogen, hydroxy, oxo, thiocarbonyl, carboxy, arylthio, thiol, alkylthio, aryloxy, heteroaryloxy, aminosulfonyl, —COOR₂ (wherein R₂ is as defined earlier), —NHC(═O)R₁₁, —NRₓR_{y}, —C(═O)NRₓR_{y}, —NHC(═O)NRₓR_{y}, —C(═O)heteroaryl,

C(═O)heterocyclyl, —OC(═O)NRₓR_{y} (where in Rₓ and R_{y} are as defined earlier), nitro, —S(O)ₙR₃ (wherein n and R₃ are as defined earlier). Unless otherwise constrained by the definition, all substituents may be further substituted by 1-3 substituents chosen from alkyl, carboxy, —COOR₂ (wherein R₂ is as defined earlier), —NLR_{y}, —C(═O)NRₓR_{y}, —O—C(═O)NRₓR_{y}, —NHC(═O)NRₓR_{y} (wherein Rₓ and R_{y} are as defined earlier), hydroxy, alkoxy, halogen, CF₃, cyano, and —S(O)ₙR₃(where R₃ and n are as defined earlier). Alkylene groups may also be interrupted by 1-5 atoms of groups independently chosen from oxygen, sulfur and —NRₐ (where Rₐ is same as defined earlier). Unless otherwise constrained by the definition, all substituents may be further substituted by 1-3 substituents chosen from alkyl, carboxy, —COOR₂ (wherein R₂ is as defined earlier), —NRₓR_{y}, —C(═O)NRₓR_{y}, —OC(═O)NRₓR_{y} (wherein Rₓ and R_{y} are as defined earlier), hydroxy, alkoxy, halogen, CF₃, cyano, and —S(O)ₙR₃ (where n and R₃ are as defined earlier).

The term “alkoxy” denotes the group O-alkyl wherein alkyl is the same as defined above.

The term “aryl” herein refers to a carbocyclic aromatic group, for example, phenyl, biphenyl or naphthyl ring and the like optionally substituted with 1 to 5 substituents selected from the group consisting of halogen (F, Cl, Br, I), hydroxy, alkyl, alkenyl, alkynyl, cycloalkyl, heterocyclyl, heteroaryl, heterocyclylalkyl or heteroarylalkyl, alkoxy, aryloxy, —CF₃, nitro, —NRₓR_{y}, acyl, cyano, acylamino, thiocarbonyl, substituted thiocarbonyl, —C(═O)NRₓR_{y}, —C(═NOH)NH₂, —NHC(═O)NRₓR_{y}, —OC(═O)NRₓR_{y} (wherein Rₓ and R_{y} are the same as defined earlier), carboxy, —S(O)ₙR₃ (where R₃ and n are the same as defined earlier), —COOR₂ (wherein R₂ is the same as defined earlier), —NHC(═O)ORₓ, —NHORₓ. The aryl group may also be fused with a heterocyclic ring, heteroaryl, and cycloalkane.

The term “aralkyl” refers to aryl linked through alkyl (wherein alkyl is the same as defined above) portion and the said alkyl portion contains carbon atoms from 1-6 and aryl is as defined above.

The term “cycloalkyl” refers to cyclic alkyl groups containing 3 to 20 carbon atoms having a single cyclic ring or multiple condensed rings, which may optionally contain one or more olefinic bonds, unless otherwise constrained by the definition. Such cycloalkyl groups include, by way of example, single ring structures such as cyclopropyl, cyclobutyl, cyclopentyl, cyclohexyl, cycloheptyl, cyclooctyl, cyclopentenyl, and the like, or multiple ring structures such as adamantanyl, and bicyclo [2.2.1] heptane, or cyclic alkyl groups to which is fused with an aryl group, for example indane or tetrahydro-naphthalene and the like. It may further be substituted with one or more substituents selected from the group consisting of alkyl, alkenyl, alkynyl, alkoxy, cycloalkyl, acyl, acylamino, —NHC(═O)ORₓ, —NHORₓ (wherein Rₓ is same as defined earlier) acyloxy, azido, cyano, halogen, hydroxy, thiocarbonyl, substituted thiocarbonyl, carboxy, —COOR₂ (wherein R₂ is the same as defined earlier), thiol, aryl, aralkyl, aryloxy, —NRₓR_{y}, —NHC(═O)NRₓR_{y}, —C(═O)NRₓR_{y}, —OC(═O)NRₓR_{y} (wherein Rₓ and R_{y} are the same as defined earlier), nitro, heterocyclyl, heteroaryl, heterocyclylalkyl, heteroarylalkyl, —CF₃, —S(O)ₙR₃ (wherein R₃ and n are the same as defined earlier). Unless otherwise constrained by the definition, all substituents may optionally be further substituted by 1-3 substituents chosen from alkyl, carboxy, hydroxy, alkoxy, halogen, CF₃, —NRₓR_{y}, —C(═O)NRₓR_{y}, —NHC(═O)NRₓR_{y}, —OC(═O)NRₓR_{y} (wherein Rₓ and R_{y} are the same as defined earlier), cyano and —S(O)ₙR₃(where R₃ and n are the same as defined earlier).

The term “carboxy” as defined herein refers to —C(═O)OH.

The term “aryloxy” denotes the group O-aryl, wherein aryl is as defined above.

The term “heteroaryl” unless otherwise specified refers to monocyclic aromatic ring structure containing 5 or 6 carbon atoms, a bicyclic or a tricyclic aromatic group having 8 to 10 carbon atoms, wherein any one or more carbon atoms of the ring are replaced with one or more heteroatom(s) independently selected from the group consisting of N, O and S optionally substituted with 1 to 3 substituent(s) selected from the group consisting of halogen (F, Cl, Br, I), hydroxy, alkoxy, alkyl, alkenyl, alkynyl, cycloalkyl, heterocyclyl, aryl, aralkyl, heteroaryl, heterocyclylalkyl, heteroarylalkyl, acyl, acylamino, thiocarbonyl, substituted thiocarbonyl, —NHC(═O)ORₓ, —NHORₓ, carboxy, —S(O)ₙR₃ (where R₃ and n are the same as defined earlier), —CF₃, —COOR₂ (wherein R₂ is the same as defined earlier), cyano, nitro, —NRₓR_{y}, —C(═O)NRₓR_{y}, —NHC(═O)NRₓR_{y} and —OC(═O)NRₓR_{y} (wherein Rₓ and R_{y} are the same as defined earlier). Examples of heteroaryl groups are pyridinyl, pyridazinyl, pyrimidinyl, pyrrolyl, oxazolyl, thiazolyl, thienyl, isoxazolyl, triazinyl, furanyl, pyrazolyl, imidazolyl, benzimidazolone, pyrazolone, benzofuranyl, indolyl, benzothiazolyl, xanthene, benzoxazolyl, and the like.

The term “heterocyclyl” unless otherwise specified refers to a non aromatic monocyclic, bicyclic (fused, bridged, or spiro) or tricyclic cycloalkyl group having 5 to 10 atoms in which 1 to 3 carbon atoms in a ring are replaced by heteroatoms selected from the group comprising of O, S and N, and are optionally benzofused or fused heteroaryl of 5-6 ring members and the said heterocyclyl group is optionally substituted wherein the substituents are selected from the group consisting of halogen (F, Cl, Br, I), hydroxy, alkoxy, alkyl, alkenyl, alkynyl, cycloalkyl, heterocyclyl, aryl, heteroaryl, aralkyl, heterocyclylalkyl, heteroarylalkyl, acyl, acylamino, thiocarbonyl, substituted thiocarbonyl, cyano, —NHC(═O)ORₓ, —NHORₓ, nitro, —CF₃, carboxy, —S(O)ₙR₃ (where R₃ and n are the same as defined earlier), —COOR₂ (wherein R₂ is the same as defined earlier), —NHC(—O)NRₓR_{y}, —C(═O)NRₓR_{y}, —OC(═O)NRₓR_{y} (wherein Rₓ and R_{y} are the same as defined earlier). Examples of heterocyclyl groups are tetrahydrofuranyl, dihydrofuranyl, dihydropyridinyl, isoxazolinyl, piperidinyl, morpholine, piperazinyl, dihydrobenzofuryl, azabicyclohexyl, azabicyclooctyl, dihydroindolyl, and the like.

The term “heteroarylalkyl” refers to heteroaryl (wherein heteroaryl is same as defined earlier) linked through alkyl (wherein alkyl is the same as defined above) portion and the said alkyl portion contains carbon atoms from 1-6.

The term “heterocyclylalkyl” refers to heterocyclyl (wherein heterocyclyl is same as defined earlier) linked through alkyl (wherein alkyl is the same as defined above) portion and the said alkyl portion contains carbon atoms from 1-6.

The term “acyl” refers to —C(═O)R″ wherein R″ is selected from the group hydrogen, alkyl, cycloalkyl, aryl, aralkyl, heteroaryl, heterocyclyl, heteroarylalkyl or heterocyclylalkyl.

The term “thiocarbonyl” refers to —C(═S)H.

The phrase “substituted thiocarbonyl” refers to —C(═S)R″, wherein R″ is selected from alkyl, cycloalkyl, aryl, aralkyl, heteroaryl, heterocyclyl, heteroarylalkyl or heterocyclylalkyl, amine or substituted amine.

The term “leaving group” generally refers to groups that exhibit the desirable properties of being labile under the defined synthetic conditions and also, of being easily separated from synthetic products under defined conditions. Examples of such leaving groups includes but not limited to halogen (F, Cl, Br, I), triflates, tosylate, mesylates, alkoxy, thioalkoxy, hydroxy radicals and the like.

The term “Protecting Groups” is used herein to refer to known moieties, which have the desirable property of preventing specific chemical reaction at a site on the molecule undergoing chemical modification intended to be left unaffected by the particular chemical modification. Also the term protecting group, unless otherwise specified may be used with groups such as hydroxy, amino, carboxy and example of such groups are found in T. W. Greene and P. G. M. Wuts, “Protective Groups in Organic Synthesis”, 2^{nd} Edn. John Wiley and Sons, New York, N.Y., which is incorporated herein by reference. The species of the carboxylic protecting groups, amino protecting groups or hydroxy protecting group employed is not so critical so long as the derivatised moiety/moieties is/are stable to conditions of subsequent reactions and can be removed at the appropriate point without disrupting the remainder of the molecule.

The term “pharmaceutically acceptable salts” refers to derivatives of compounds that can be modified by forming their corresponding acid or base salts. Pharmaceutically acceptable salts may also be formed by complete derivatization of the amine moiety e.g., quaternary ammonium salts.

In accordance with a second aspect, provided are methods for the treatment or prophylaxis of an animal or a human suffering from a disease or disorder of the respiratory, urinary and gastrointestinal systems, wherein the disease or disorder is mediated through muscarinic receptors. The methods include administration of at lest one compound having the structure of Formula I.

In accordance with a fourth aspect, provided are methods for the treatment or prophylaxis of an animal or a human suffering from a disease or disorder of the respiratory system, such as bronchial asthma, chronic obstructive pulmonary disorders (COPD), pulmonary fibrosis, and the like; urinary system which induce disorders as urinary incontinence, lower urinary symptoms (LUTS), etc.; and gastrointestinal system, such as irritable bowel syndrome, obesity, diabetes and gastrointestinal hyperkinesis, with one or more compounds of Formula I, wherein the disease or disorder is associated with muscarinic receptors.

In accordance with fifth aspect, provided are processes for preparing the compounds of Formula I.

The compounds described herein can exhibit significant potency in terms of their activity, as determined by in vitro receptor binding and functional assays and in vivo experiments using anaesthetized rabbits. The compounds that were found active in vitro were tested in vivo. Some of the compounds are potent muscarinic receptor antagonists with high affinity towards M₁ and M₃ receptors than M₂ and/or M₅ receptors. Therefore, pharmaceutical composition for the treatment of the disease or disorders associated with muscarinic receptors are provided. In addition, the compounds can be administered by any route of administration, including orally or parenterally.

The compounds disclosed herein may be prepared by methods represented by the reaction sequences, for example, as generally shown in Schemes I:

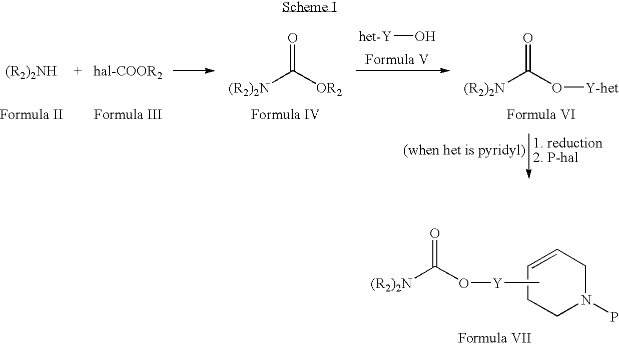

Compounds of Formula VI and Formula VII can be prepared following the procedure as described in scheme I. The preparation comprises condensing a compound of Formula II (wherein R₂ is the same as defined earlier) with compound of Formula III (wherein hal is Cl, Br, I) to give compound of Formula IV (wherein R_{z} is alkyl or aryl), which is reacted with compound of Formula V (wherein Y and het are the same as defined earlier) to give a compound of Formula VI, which undergoes reduction (when het is pyridyl) followed by reaction with P-hal (wherein P is selected from alkyl, cycloalkyl, heteroaryl, heterocyclyl, heteroarylalkyl, heterocyclylalkyl, aralkyl, —C(═O)OC(CH₃)₃, —C(═O)OC(CH₃)₂CHBr₂ or —C(═O)OC(CH₃)₂CCl₃) to give a compound of Formula VII.

The reaction of a compound of Formula II with compound of Formula III to give the compound of Formula IV can be carried out in an organic solvent (for example, tetrahydrofuran, dioxane, dimethylformamide, diethylether or dichloromethane) in the presence of a base (for example, triethylamine, pyridine or diisopropylethylamine).

The transesterification of a compound of Formula IV with compound of Formula V to give a compound of Formula VI can be carried out in an organic solvent (for example, toluene, heptane, dimethylformamide or xylene) in the presence of a base (for example, sodium hydride, lithiumdiisopropylamide or pyridine).

The reduction of a compound of Formula VI (when het is pyridyl) can be carried out with sodium borohydride, di-isobutyl aluminium hydride or lithium borohydride followed by reaction with a compound of Formula P-hal in an organic solvent ethanol, methanol or isopropyl alcohol to give a compound of Formula VII.

Examples of compounds include:
(1-Benzyl-1H-imidazol-2-yl)methyl benzyl(phenyl)carbamate (Compound No. 1);
2-(2-Methyl-1H-imidazol-1-yl)ethyl benzyl(phenyl)carbamate (Compound No. 2);
2-(2-Methyl-1H-imidazol-1-yl)ethylphenyl[4-trifluoromethyl)benzyl]carbamate (Compound No. 3);
(1-Benzyl-1H-imidazol-2-yl)methylphenyl[4-(trifluoromethyl)benzyl carbamate] (Compound No. 4);
2-(1H-Imidazol-1-yl)ethyl benzyl(phenyl)carbamate (Compound No. 5);
(1-Benzyl-1,2,3,6-tetrahydropyridin-4-yl)methyl benzyl(phenyl)carbamate (Compound No. 6);
Pyridin-3-ylmethyl phenyl[4-trifluoromethyl)benzyl]carbamate (Compound No. 7);
Pyridin-3-ylmethyl benzyl(phenyl)carbamate (Compound No. 8);
Pyridin-2-ylmethyl benzyl(phenyl)carbamate (Compound No. 9);
(1-Benzyl-1,2,5,6-tetrahydropyridin-3-yl)methyl benzyl(phenyl)carbamate (Compound No. 10);
Pyridin-4-ylmethyl benzyl(phenyl)carbamate (Compound No. 11);
2-(1H-Imidazol-1-yl)ethylphenyl[4-(trifluoromethyl)benzyl]carbamate (Compound No. 12).

or its pharmaceutically accepted salts, pharmaceutically acceptable solvates, enantiomers, diastereomers, polymorphs or N-oxides.

In the above schemes, where specific bases, condensing agents, protecting groups, deprotecting agents, solvents, catalysts, temperatures, etc. are disclosed, it is to be understood that other bases, condensing agents, protecting groups, deprotecting agents, solvents, catalysts, temperatures, etc. known to those skilled in the art may be used. Similarly, the reaction conditions, such as temperature and duration, may be adjusted accordingly.

Suitable salts of the compounds represented by the Formula I were prepared so as to solubilize the compound in aqueous medium for biological evaluations, as well as to be compatible with various dosage formulations and/or aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, tolounesulphonate and methanesulphonate). When carboxyl groups are present as substituents in the compounds described herein, they may be present in the form of an alkaline or alkali metal salt (for example, sodium, potassium, calcium, magnesium, and the like). These salts may be prepared by various techniques, such as treating the compound with an equivalent amount of inorganic or organic, acid or base in a suitable solvent.

The compounds described herein include their enantiomers, diastereomers, N-oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with one or more pharmaceutically acceptable carrier, excipient or diluents are also provided.

Where desired, the compounds of Formula I and/or their pharmaceutically acceptable salts, pharmaceutically acceptable solvates, stereoisomers, tautomers, racemates, prodrugs, metabolites, polymorphs or N-oxides may be advantageously used in combination with one or more other therapeutic agents. Examples of other therapeutic agents include, but are not limited to, corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

The compositions can be administered by route of administration, including, for example, inhalation, insufflation, orally, rectally, parenterally (intravenously, intramuscularly or subcutaneously), intracisternally, intravaginally, intraperitoneally or topically.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients. The compositions can be administered by the nasal respiratory route for local or systemic effect. Compositions can be nebulized by use of inert gases. Nebulized solutions may be inhaled directly from a nebulizing device or the nebulizing device can be attached to a face masks tent, or intermittent positive pressure breathing machine. Solution, suspension, or powder compositions can be administered nasally from devices.

Solid dosage forms for oral administration may be presented in discrete units, for example, capsules, cachets, lozenges, tablets, pills, powders, dragees or granules, each containing a predetermined amount of the one or more active compound (i.e., at least a compound described herein). In such solid dosage forms, the active compound can be admixed with one or more inert excipient (or carrier or diluents), such as sodium citrate or dicalcium phosphate or (a) fillers or extenders, for example, starches, lactose, sucrose, glucose, mannitol and silicic acid, (b) binders, for example, carboxymethylcellulose, alignates, gelatin, polyvinylpyrrolidone, sucrose and acacia, (c) humectants, for example, glycerol, (d) disintegrating agents, for example, agar-agar, calcium carbonate, potato or tapioca starch, alginic acid, certain complex silicates and sodium carbonate, (e) solution retarders, for example paraffin, (f) absorption accelerators, for example, quaternary ammonium compounds, (g) wetting agents, for example, cetyl alcohol and glycerol monostearate, (h) adsorbents, for example, kaolin and bentonite, and (i) lubricants, for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type also include soft and hard-filled gelatin capsules using such excipients, for example lactose or milk sugar, as well as high molecular weight polyethylene glycols, and the like.

Solid dosage forms can be prepared with one or more coatings and shells, such as enteric coatings and others well known in this art. Solid dosage forms may contain opacifying agents, and formulated to release one or more active compounds in a specific part of the gastrointestinal tract, i.e., in a controlled delayed manner. Examples of embedding compositions, which can be used, include polymeric substances and waxes.

Active compounds can also be in micro-encapsulated form, if appropriate, with one or more carriers, excipients, diluents.

Liquid dosage forms for oral administration include pharmaceutically acceptable emulsions, solutions, suspensions, syrups and elixirs. Liquid dosage forms may contain one or more inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan and the like or mixtures thereof.

Such composition described herein can also include one or more adjuvants, for example, wetting agents, emulsifying and suspending agents, sweetening, flavoring and perfuming agents, colorants or dyes.

Suspensions may contain one or more suspending agents, for example, ethoxylated isostearyl alcohols, polyoxyethylene sorbitol and sorbitan esters, microcrystalline cellulose, aluminium metahydroxide, bentonite, agar-agar and tragacanth, or mixtures of these substances, and the like.

Dosage forms for topical administration include powder, spray, inhalant, ointment, creams, salve, jelly, lotion, paste, gel, aerosol, or oil. Active component can be admixed under sterile conditions with one or more pharmaceutically acceptable carrier, excipients or diluents and optionally one or more preservatives, buffers or propellants. Opthalmic formulations, eye ointments, powders and solutions are also encompassed herein.

Compositions suitable for parenteral injection may comprise pharmaceutically acceptable sterile aqueous or nonaqueous solutions, dispersions, suspensions or emulsions and sterile powders for reconstitution into sterile injectable solutions or dispersions. These preparations may contain anti-oxidants, buffers, bacteriostats and solutes, which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating (e.g., lecithin), by the maintenance of the required particle size in the case of dispersions and by the use of surfactants.

Such compositions may also contain adjuvants, such as preserving, wetting, emulsifying and dispensing agents. Various antibacterial and antifungal agents, for example, parabens, chlorobutanol, phenol, sorbic acid, and the like, may be used, in particular to prevent microorganism activity. Such composition may also include isotonic agents, for example sugars, sodium chloride and the like. Prolonged absorption of the injectable composition can be facilitated by the use of agents delaying absorption, for example, aluminum monosterate and gelatin.

Suppositories for rectal administration can be prepared by mixing the active ingredients with one or more suitable nonirritating excipient, such as cocoa butter and polyethylene glycols or a suppository wax, which are solid at ordinary temperatures but liquid at body temperature and which melt in the rectum or vaginal cavity and release the active ingredients.

Compounds described herein can be incorporated into slow release or targeted delivery systems, such as polymer matrices, liposomes, and microspheres. The compounds may be sterilized, for example, by filtration through a bacteria-retaining filter, or by incorporating sterilizing agents in the form of sterile solid compositions, which can be dissolved in sterile water, or some other sterile injectable medium immediately before use.

Actual dosage levels of active ingredient in the compositions described herein and administration schedules of individual dosages may be readily varied to provide an effective amount of active ingredient that facilitate a desired therapeutic response for a particular composition and method of administration. It is to be understood, however, that the specific dose level for any particular patient can depend upon a variety of factors including for example, the body weight, general health, sex and diet of the patient; specific compound chosen; route of administration; the desired duration of treatment; rates of absorption and excretion; combination with other drugs and the severity of the particular disease being treated and is ultimately at the discretion of the physician.

The pharmaceutical compositions described herein can be produced and administered in dosage units, each unit containing a therapeutically amount of one or more compound described herein and/or at least one physiologically acceptable addition salt thereof. The dosage may be varied over wide limits as the compounds can be effective at low dosage levels and relatively free of toxicity. The compounds may be administered in the low micromolar concentration, which amounts are therapeutically effective, and the dosage may be increased accordingly up to the maximum dosage tolerated by the patient.

While the present invention has been described in terms of its specific embodiments, certain modification and equivalents will be apparent to those skilled in the art and are included within the scope of the present invention. The examples are provided to illustrate particular aspects of the disclosure and do not limit the scope of the present invention as defined by the claims.

### EXAMPLES

### General Procedure

### Synthesis of N-Benzyl Aniline

To a solution of benzaldehyde (5 g, 47.1 mmol) and aniline (4.8 g, 51.8 mmol) in dichloromethane at room temperature under nitrogen atmosphere was added sodium triacetoxy borohydride (9.9 g, 141.3 mmol) in small portions and stirred the reaction mixture at room temperature overnight. The reaction mixture was concentrated under reduced pressure and quenched with potassium hydroxide solution (10%). The aqueous layer was extracted with ethyl acetate, dried over anhydrous sodium sulphate and concentrated under reduced pressure. The residue thus obtained was purified by column chromatography using 2% ethyl acetate in hexane to furnish the title compound. Yield: 4.3 g.

¹HNMR (CDCl₃) δ: 4.30 (s, 2H), 6.60-6.72 (m, 3H), 7.14-7.36 (m, 7H).

### Synthesis of 4-nitrophenyl benzyl(phenyl)carbamate

To a solution of N-benzyl aniline (2 g, 10.9 mmol) in dichloromethane was added triethylamine (4.56 mL, 32.7 mmol) and p-nitrochloroformate (2.8 g, 14.2 mmol) and stirred the reaction mixture overnight. The reaction mixture was washed with ammonium chloride and extracted with dichloromethane. The organic solvent was washed with brine, dried over anhydrous sodium sulphate and concentrated under reduced pressure. The residue thus obtained was purified by column chromatography using 4% ethylacetate in hexane solvent mixture as eluent to furnish the title compound. Yield: 3 g.

Mass spectrum (m/z, +ve ion mode): 349 (M⁺1);

### Synthesis of 2-(2-methyl-imidazol-1-yl)-ethanol

### Step a: (2-Bromo-ethoxy)-tert-butyl-dimethyl-silane

To a solution of 2-bromoethanol (5 g, 40 mmol) in dimethylformamide (25 mL) was added tert-butydimethylsilyl chloride (7.29 g, 48 mmol) and imidazole (6.86 g, 100 mmol). The reaction mixture was stirred overnight followed by quenching with water and extraction with ethyl acetate. The organic layer was dried over anhydrous sodium sulphate and concentrated under reduced pressure to furnish the title compound. Yield: 8 g.

### Step b: 1-[2-Tert-butyl-dimethyl-silanyloxy)-ethyl]-2-methyl-1H-imidazole

Sodium hydride (8.4 g, 210 mmol) was added slowly to dry dimethylformamide (40 mL) precooled at −10° C. under nitrogen atmosphere. To the resulting suspension was added 2-methyl imidazole (20.67 g, 252 mmol) at −10° C. and the reaction mixture was allowed to warm to room temperature. The reaction mixture was stirred for 1 hour at room temperature followed by cooling to 0° C. To the mixture was added solution of the compound obtained from step a above (20 g, 84 mmol) in dimethylformamide (10 mL) and stirred at room temperature for overnight. The mixture was quenched with aqueous ammonium chloride solution and extracted with dichloromethane. The dichloromethane layer was dried over anhydrous sodium sulphate and concentrated under reduced pressure to furnish the title compound. Yield: 12.5 g.

### Step c: 2-(2-Methyl-imidazol-1-yl)-ethanol

To a compound obtained from step b above (12.5 g, 55.6 mmol) was added a solution of ethanolic hydrochloric acid solution (2%, 90 mL) at room temperature and stirred the mixture overnight. The reaction mixture was concentrated under reduced pressure. The residue thus obtained was washed with diethyl ether to furnish the title compound. Yield: 3.9 g.

¹H NMR (CD₃OD) δ: 7.58-7.55 (d, 1ArH), 7.43-7.40 (d, 1ArH), 4.26-4.24 (t, 2H), 3.90-3.87 (t, 21-1), 2.67 (s, 3H)

Following compound was prepared similarly, by using imidazole in place of 2-methyl imidazole in step b.

### 2-(Imidazol-1-yl)-ethanol

¹H NMR (D₂O) δ: 8.63 (s, 1ArH), 7.41 (s, 1ArH), 7.35 (s, 1ArH), 4.24-4.22 (t, 2H), 3.82-3.80 (t, 2H).

### Synthesis of (1-benzyl-1H-imidazol-2-yl)methanol

### Step a: 1-Benzyl-1H-imidazole-2-carbaldehyde

To a solution of the compound 1H-imidazole-2-carboxaldehyde (5 g, 52.0 mmol) in methanol (20 mL) was added acetone (40 mL), potassium carbonate (21.56 g, 156.2 mmol) and tetrabutyl ammonium bromide (catalytic amount). The reaction mixture was stirred at room temperature for 1 hour followed by the addition of benzyl bromide (8.9 g, 52.0 mmol). The reaction mixture was stirred at room temperature for 4 hours. The mixture was filtered through sintered funnel and the filtrate was concentrated under reduced pressure. The residue thus obtained was purified by column chromatography using 0.5% methanol in dichloromethane to furnish the title compound.

### Step b: (1-Benzyl-1H-imidazol-2-yl)methanol

A solution of the compound obtained from step a above (5 g, 26.8 mmol) in methanol (20 mL) was cooled at 0° C. followed by the addition of sodium borohydride portionwise and stirred the reaction mixture at room temperature for 5 hours. The solvent was evaporated under reduced pressure and the residue thus obtained was dissolved in ether, washed with water and brine, dried over anhydrous sodium sulphate and concentrated under reduced pressure to furnish the title compound. Yield 2 g.

¹HNMR (CDCl₃) δ: 4.64 (s, 2H), 5.23 (s, 2H), 6.83 (s, 1H), 6.91 (s, 1H), 71.4-7.37 (m, 7H)

### Example 1

### Synthesis of pyridin-2-ylmethyl benzyl(phenyl)carbamate (Compound No. 9)

A solution of the pyridine-2-methanol (commercially available) (281 mg, 2.58 mmol), sodium hydride (62 mg) and toluene (20 mL) was stirred for 5 minutes. To the resulting reaction mixture was added 4-nitrophenyl benzyl(phenyl)carbamate (600 mg, 1.72 mmol) and stirred the mixture at 130-140° C. for 4 hours. The organic solvent was evaporated under reduced pressure and the reaction mixture was quenched with ammonium chloride solution. The mixture was extracted with ethyl acetate, washed the organic layer with brine and water and concentrated under reduced pressure. The residue thus obtained was purified by column chromatography using 40% ethyl acetate in hexane as eluent to furnish the title compound. Yield: 80 mg.

¹H NMR (CDCl₃) δ: 8.53 (s, 1ArH), 7.60-7.56 (m, 1ArH), 7.23-7.16 (m, 11ArH), 7.06-7.04 (m, 1ArH), 5.29 (s, 2H), 4.90 (s, 2H).

Mass spectrum (m/z, +ve ion mode): 319 (M⁺1);

Following compounds were prepared similarly,

### Pyridin-3-ylmethyl benzyl(phenyl)carbamate (Compound No. 8),

Mass spectrum (m/z, +ve ion mode): 319 (M⁺1);

### Pyridin-4-ylmethyl benzyl(phenyl)carbamate (Compound No. 11),

Mass spectrum (m/z, +ve ion mode): 319 (M⁺1);

### Pyridin-3-ylmethyl phenyl[4-(trifluoromethyl)benzyl]carbamate (Compound No. 7),

Mass spectrum (m/z, +ve ion mode): 387 (M⁺1).

### Example 2

### Synthesis of (1-benzyl-1H-imidazol-2-yl)methyl benzyl(phenyl)carbamate (Compound No. 1)

To a solution of the 4-nitrophenyl benzyl(phenyl)carbamate (400 mg, 1.14 mmol) in dimethylformamide (15 mL) was added (1-benzyl-1H-imidazol-2-yl)methanol (324 mg, 1.72 mmol) and sodium hydride (68.96 mg, 1.72 mmol). The reaction mixture was stirred for 2-3 hours. The mixture was subsequently diluted with water and extracted with ethyl acetate. The organic layer was washed with water and brine, dried over anhydrous sodium sulphate, filtered and concentrated under reduced pressure. The residue thus obtained was purified by column chromatography using 4% methanol in dichloromethane to furnish the title compound.

¹H NMR (CDCl₃) δ: 7.28-7.22 (m, 8ArH), 7.20-7.15 (m, 3ArH), 7.02-6.80 (m, 6ArH), 5.21 (s, 21-1), 4.94 (s, 2H), 4.77 (s, 2H);

Mass spectrum (ink, +ve ion mode): 398 (M⁺1).

Following compounds were prepared similarity,

### 2-(2-Methyl-1H-imidazol-1-yl)ethyl benzyl(phenyl)carbamate Compound No. 2),

Mass spectrum (m/z, +ve ion mode): 336 (M⁺1);

### 2-(2-Methyl-1H-imidazol-1-yl)ethylphenyl[4-(trifluoromethyl)benzyl]carbamate (Compound No. 3),

Mass spectrum (m/z, +ve ion mode): 404 (M⁺1);

### (1-Benzyl-1H-imidazol-2-yl)methyl phenyl[4-(trifluoromethyl)benzyl carbamate] (Compound No. 4),

Mass spectrum (m/z, +ve ion mode): 466 (M⁺1);

### 2-(1H-Imidazol-1-yl)ethyl benzyl(phenyl)carbamate Compound No. 5

Mass spectrum (m/z, +ve ion mode): 322 (M⁺1);

### 2-(1H-Imidazol-1-yl)ethyl phenyl[4- trifluoromethyl)benzyl]carbamate (Compound No. 12),

Mass spectrum (m/z, +ve ion mode): 390 (M+1).

### Example 3

### Synthesis of (1-Benzyl-1,2,5,6-tetrahydropyridin-3-yl)methyl benzyl(phenyl)carbamate (Compound No. 10)

To a solution of the Compound No. 8 (180 mg, 0.5 mmol) in ethanol (10 mL) was added benzyl bromide (1.5 eq.) and refluxed the reaction mixture for 18 hours. The reaction mixture was cooled to room temperature. The solvent was evaporated under reduced pressure followed by the addition of sodium borohydride (45 mg, 1.1 mmol) and ethanol. The reaction mixture was stirred overnight. The mixture was concentrated under reduced pressure and the residue thus obtained was purified by column chromatography using 30% ethyl acetate in hexane to furnish the title compound. Yield: 50 mg.

¹H NMR (CDCl₃) δ: 7.32-7.08 (m, 15ArH), 5.70 (s, 1H), 4.83 (s, 2H), 4.51 (s, 2H), 3.45 (s, 2H), 2.80 (s, 2H), 2.49-2.47 (m, 2H), 2.17-2.14 (m, 2H).

Mass spectrum (m/z, +ve ion mode): 413 (M⁺1);

Following compound was prepared similarly,

### (1-Benzyl-1,2,3,6-tetrahydropyridin-4-yl)methyl benzyl(phenyl)carbamate (Compound No. 6),

Mass spectrum (m/z, +ve ion mode): 413 (M⁺1);

### Biological Activity

### In-Vitro Experiments

### Radioligand Binding Assays:

The affinity of test compounds for M₂ and M₃ muscarinic receptor subtypes was determined by [³H]-N-Methylscopolamine (NMS) binding studies using rat heart and submandibular gland respectively as described by Moriya et al., Life Sci, 1999, 64(25): 2351-2358 with minor modifications. Specific binding of [³H]-NMS was also determined using membranes from Chinese hamster ovary (CHO) cells expressing cloned human muscarinic receptor subtypes.

### Membrane Preparation:

### (a) Rat Tissues

Submandibular glands and heart were isolated and placed in ice-cold homogenizing buffer (HEPES 20 mM, 10 mM EDTA, pH 7.4) immediately after sacrifice. The tissues were homogenized in ten volumes of homogenizing buffer and the homogenate was filtered through two layers of wet gauze and filtrate was centrifuged at 500 g for 10 min. The supernatant was subsequently centrifuged at 40,000 g for 20 min. The pellet thus obtained was resuspended in assay buffer (HEPES 20 mM, EDTA 5 mM, pH 7.4) and were stored at −70° C. until the time of assay.

### (b) CHO Cells Expressing Human Recombinant Receptors

The cell pellets were homogenised for 30 sec at 12,000 to 14,000 rpm, with intermittent gaps of 10-15 sec in ice-cold homogenising buffer (20 mM HEPES, 10 mM EDTA, pH 7.4). The homogenate was then centrifuged at 40,000 g for 20 min at 4° C. The pellet thus obtained was resuspended in homogenising buffer containing 10% sucrose and was stored at −70° C. until the time of assay.

### Ligand Binding Assay:

The test compounds were dissolved and diluted in dimethylsulphoxide. The membrane homogenates (5-10 μg protein) were incubated in 250 μL of assay buffer (20 mM HEPES, pH 7.4) at 24-25° C. for 3 hrs. Non-specific binding was determined in the presence of 1 μM Atropine. The incubation was terminated by vacuum filtration over GF/B fibre filter mats (Wallac) using Skatron cell harvester. The filters were then washed with ice-cold 50 mM Tris HCl buffer (pH 7.4). The filter mats were dried and transferred to 24 well plates (PET A No Cross Talk) followed by addition of 500 μl of scintillation cocktail. Radioactivity retained on filters was counted in Microbeta scintillation counter. The IC₅₀ & Kd were estimated by using the non-linear curve-fitting program using GraphPad Prism software. The value of inhibition constant, Ki were calculated from competitive binding studies by using Cheng & Prusoff's equation (Biochem Pharmacol, 22: 3099-3108 (1973)), Ki=IC₅₀/(1+[L]/Kd), where [L] is the concentration of ligand [³H]-N-methyl scopolamine used in the particular experiment and Kd is the estimate of affinity of receptors to the ligand.

The above disclosed compounds exhibited Ki values for M₃ and M₂ receptors in the nanomolar to micromolar range. More particularly, the Ki values for M₃ and M₂ receptors were in the ranges of 1 nM to about 10 μM and 10 nM to about 10 μM, respectively.

### Functional Experiments Using Isolated Rat Bladder:

### Methodology:

Animals were euthanized by overdose of thiopentone and whole bladder was isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder was cut into longitudinal strips (3 mm wide and 5-6 mm long) and mounted in 10 mL organ baths at 30° C., with one end connected to the base of the tissue holder and the other end connected through a force displacement transducer. Each tissue was maintained at a constant basal tension of 1 g and allowed to equilibrate for 1.5 hour during which the Tyrode buffer was changed every 15-20 min. At the end of equilibration period, the stabilization of the tissue contractile response was assessed with 1 μmol/L of carbachol till a reproducible response is obtained. Subsequently a cumulative concentration response curve to carbachol (10⁻⁹ mol/L to 3×10⁻⁴ mol/L) was obtained. After several washes, once the baseline is achieved, cumulative concentration response curve was obtained in presence of NCE (NCE added 20 min. prior to the second cumulative response curve.

The contractile results were expressed as % of control E max. ED₅₀ values were calculated by fitting a non-linear regression curve (Graph Pad Prism). pKb values were calculated by the formula pKb=−log [(molar concentration of antagonist/(dose ratio−1))]

where,
dose ratio=ED₅₀ in the presence of antagonist/ED₅₀ in the absence of antagonist.

### In-Vitro Functional Assay to Evaluate Efficacy of MRA on Guinea Pig & Rat Trachea Animals and Anaesthesia

Trachea tissue is obtained from guinea pigs (under an overdose of anesthesia (sodium pentobarbital, ˜300 mg/kg i.p) and immediately kept in an ice-cold Krebs Henseleit buffer of the following composition (mM): NaCl, 118; KCl 4.7; CaCl₂, 2.5; MgSO₄, 1.2; NaHCO₃, 25; KH₂PO₄, 1.2, glucose 11.1.

### Trachea Experiments:

Trachea tissue is cleaned off adherent fascia and cut into seven to eight strips of equal size (with approximately 4-5 tracheal rings in each strip). The trachea is opened along the mid-dorsal surface with the smooth muscle band intact and a series of transverse cuts from alternate sides is made so that they did not transect the preparation completely. The opposite end of the cut rings are tied using thread. The tissue is mounted in isolated tissue baths containing 10 mL Krebs Henseleit buffer maintained at 37° C. and bubbled with carbogen (95% oxygen and 5% carbon dioxide), at a basal tension of 1 gm. The buffer is changed 3-4 times for about an hour. The tissues are equilibrated for 1 hour for stabilization. After 1 hour, the tissue is contacted with 60 mM KCl. This procedure is repeated after every 2-3 washes until two similar consecutive responses are obtained. At the end of stabilization, a carbachol concent ration-response curve is performed on all the tissues. The tissues were washed until the baseline is obtained. Thereafter, each tissue was incubated with different concentrations of MRA/Standard/Vehicle for 20 minutes followed by a second cumulative dose response curve to carbachol. The contractile response of tissues is recorded either on a Powerlab system or on Grass polygraph (Model 7). The responses to carbachol were standardized as a percentage of the maximum carbachol response of the control CRC. The carbachol EC₅₀ values in the presence and absence of inhibitor are determined using graph pad prism. pK_{B} values, an index of functional antagonism from EC₅₀ data, were calculated using the following relationship:

−log [antagonist(M)/(EC₅₀ antagonist/EC₅₀ control)−1]

The data is expressed as mean±s.e.m for n observations. In tissues where Eₘₐₓ attained is less than 50%, pK_{B} is calculated by Kenakin's double reciprocal plot.

All drugs and chemicals used in the study are of AR grade. Carbachol is procured from Sigma Chemicals, U.S.A. Stock solutions of Standard/New Chemical Entities (NCEs) are prepared in DMSO. Subsequent dilutions are prepared from the stock in MilliQ water.

In-Vitro Functional Assay to Evaluate Efficacy of “MRA” in Combination with “Pde-Iv inhibitors”

### Animals and Anaesthesia:

Trachea tissue is obtained from a guinea pig (400-600 gm) under anesthesia (sodium pentobarbital, 300 mg/kg i.p) and is immediately kept in an ice-cold Krebs Henseleit buffer. Indomethacin (10 uM) is present throughout the KH buffer to prevent the formation of bronchoactive prostanoids.

### Trachea Experiments:

Trachea tissue is cleaned off adherent fascia and cut it into strips of equal size (with approx. 4-5 tracheal rings in each strip). The epithelium is removed by careful rubbing, minimizing damage to the smooth muscle. The trachea is opened along the mid-dorsal surface with the smooth muscle band intact and a series of transverse cuts is made from alternate sides so that they do not transect the preparation completely. Opposite ends of the cut rings are tied with the help of a thread. The tissue is mounted in isolated tissue baths containing 10 mL Krebs Henseleit buffer maintained at 37° C. and is bubbled with carbogen, at a basal tension of 1 gm. The buffer is changed 4-5 times for about an hour and the tissue is equilibrated for 1 hour for stabilization. After 1 hour, the tissue is contacted with 1 uM carbachol. Repeat this after every 2-3 washes until two similar consecutive responses are obtained. At the end of stabilization, the tissue is washed for 30 minutes followed by incubation with suboptimal dose of MRA/Vehicle for 20 minutes prior to contraction of the tissues with 1 μM carbachol. The relaxant activity of the PDE-IV inhibitor [10⁻⁹ M to 10⁻⁴ M] on the stabilized developed tension/response is assessed. The contractile response of tissues is recorded either on a Powerlab data acquisition system or on a Grass polygraph (Model 7). The relaxation is expressed as a percentage of maximum carbachol response. The data is expressed as mean±s.e. mean for n observations. The EC₅₀ is calculated as the concentration producing 50% of the maximum relaxation to 1 uM carbachol. The percent relaxation between the treated and control tissues is compared using non-parametric unpaired t-test. A p value of <0.05 is considered to be statistically significant.

### In-Vivo Experiments

### In-Vivo Assay to Evaluate Efficacy of MRA Inhibitors

Male Guinea pigs are anesthetized with urethane (1.5 g/kg, i.p.). Trachea is cannulated along with jugular vein (for carbachol challenge) and animals are placed in the Plethysmograph-Box (PLY 3114 model; Buxco Electronics, Sharon, USA.). Respiratory parameters are recorded using Pulmonary Mechanics Analyser, Biosystems XA software (Buxco Electronics, USA), which calculates lung resistance (R_{L}) on a breath-by-breath basis. Bronchoconstriction is induced by injections of Carbachol (10 μg/kg) delivered into the jugular vein. Increase in R_{L} over a period of 5 min post carbachol challenge is recorded in presence or absence of MRA or vehicle at 2 hrs and 12 hrs post treatment and expressed as % increase in R_{L} from basal

$$\% Inhibition = \frac{{R}_{L vehicle} - {R}_{L test}}{{R}_{L vehicle}} \times 100$$

R_{L vehicle} % increase in lung resistance from basal in vehicle treated
R_{L test} % increase in lung resistance from basal at a given dose of test
In-vivo assay to evaluate efficacy of MRA in combination with PDE-IV inhibitors Drug Treatment:

MRA (1 μg/kg to 1 mg/kg) and PDE-IV inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/mL) for 40 min. One group of vehicle treated rats is exposed to phosphate buffered saline (PBS) for 40 min. Two hours after LPS/PBS exposure, animals are placed inside a whole body plethysmograph (Buxco Electronics, USA) and exposed to PBS or increasing acetylcholine (1, 6, 12, 24, 48 and 96 mg/mL) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

where,
PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 min, at 4° C. Total leukocyte count is performed in the resuspended sample. A portion of suspension is cytocentrifuged and stained with Leishmann's stain for differential leukocyte count. Total leukocyte and Neutrophil counts are expressed as cell count (millions cells mL⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

where,
NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
The percent inhibition data is used to compute ED₅₀ vales using Graph Pad Prism software (Graphpad Software Inc., USA).
In-Vivo Assay to Evaluate Efficacy of MRA in Combination with Corticosteroids Ovalbumin Induced Airway Inflammation:

Guinea pigs are sensitised on days 0, 7 and 14 with 50-μg ovalbumin and 10 mg aluminium hydroxide injected intraperitoneally. On days 19 and 20 guinea pigs are exposed to 0.1% w v⁻¹ ovalbumin or PBS for 10 min, and with 1% ovalbumin for 30 min on day 21. Guinea pigs are treated with test compound (0.1, 0.3 and 1 mg kg⁻¹) or standard 1 mg kg⁻¹ or vehicle once daily from day 19 and continued for 4 days. Ovalbumin/PBS challenge is performed 2 hours after different drug treatment.

Twenty four hours after the final ovalbumin challenge BAL is performed using Hank's balanced salt solution (HBSS). Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and eosinophil count are expressed as cell count (millions cells mL⁻¹ of BAL). Eosinophil is also expressed as percent of total leukocyte count. % inhibition is computed using the following formula.

$$\% Inhibition = \frac{{Eos}_{OVA} - {Eos}_{TEST}}{{Eos}_{OVA} - {Eos}_{CON}} \times 100$$

where,
Eos_{OVA}=Percentage of eosinophil in untreated ovalbumin challenged group
Eos_{TEST}=Percentage of eosinophil in group treated with a given dose of test compound
Eos_{CON}=Percentage of eosinophil in group not challenged with ovalbumin.
In-Vivo Assay to Evaluate Efficacy of “MRA” in Combination with p38 Map Kinase Inhibitors
Lipopolysaccharide (LPS) induced airway hyperreactivity (AHR) and neutrophilia:

### Drug Treatment:

MRA (1 μg/kg to 1 mg/kg) and p38 MAP kinase inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/mL) for 40 min. One group of vehicle treated rats is exposed to phosphate buffered saline (PBS) for 40 min. Two hours after LPS/PBS exposure, animals are placed inside a whole body plethysmograph (Buxco Electronics, USA) and exposed to PBS or increasing acetylcholine (1, 6, 12, 24, 48 and 96 mg/mL) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

where,
PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells mL⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

where,
NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
The percent inhibition data is used to compute ED₅₀ values using Graph Pad Prism software (Graphpad Software Inc., USA).
In-Vivo Assay to Evaluate Efficacy of “MRA” in Combination with β2-Agonists

### Drug Treatment:

MRA (1 μg/kg to 1 mg/kg) and long acting β₂ agonist is instilled intratracheally under anesthesia either alone or in combination.

### Method

Wistar rats (250-350 gm) or balb/C mice (20-30 gm) is placed in body box of a whole body plethysmograph (Buxco Electronics, USA) to induce bronchoconstriction. Animals are allowed to acclimatise in the body box and are given successive challenges, each of 2 min duration, with PBS (vehicle for acetylcholine) or acetylcholine (i.e. 24, 48, 96, 144, 384, and 768 mg/mL). The respiratory parameters are recorded online using Biosystem XA software, (Buxco Electronics, USA) for 3 min. A gap of 2 min is allowed for the animals to recover and then challenged with the next higher dose of acetylcholine (ACh). This step is repeated until Penh of rats attained 2 times the value (PC-100) seen with PBS challenge. Following PBS/ACh challenge, Penh values (index of airway resistance) in each rat/mice is obtained in the presence of PBS and different doses of ACh. Penh, at any chosen dose of ACh is, expressed as percent of PBS response. The Penh values thus calculated are fed into Graph Pad Prism software (Graphpad Software Inc., USA) and using a nonlinear regression analysis PC100 (2 folds of PBS value) values are computed. % inhibition is computed using the following formula.

$$\% Inhibition = \frac{PC {100}_{TEST} - PC {100}_{CON}}{768 - PC {100}_{CON}} \times 100$$

where,
PC100_{CON}=PC100 in vehicle treated group
PC100_{TEST}=PC100 in group treated with a given dose of test compound
768=is the maximum amount of acetylcholine used.

We claim:

1. A compound of Formula 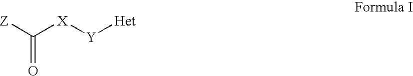
or a pharmaceutically accepted salts, pharmaceutically acceptable solvates, enantiomers, diastereomers, polymorphs or N-oxides thereof, wherein
Het is heterocyclyl or heteroaryl wherein nitrogen atom in heterocyclyl ring may also be quaternized to form a quaternary ammonium salts,
X is O, S or NR₁ (wherein R₁ is as defined below);
Y is no atom or —(CH₂)ₙ;
n is an integer from 1 to 6;
Z is —NHR₂, —N(R₂)₂ (wherein R₂ is as defined below), aryl or cycloalkyl;
R₁ is hydrogen, alkyl or aralkyl;
R₂ is independently selected from alkyl, aryl, aralkyl, heteroaryl, cycloalkyl, heterocyclyl, heterocyclylalkyl or heteroarylalkyl;

2. A compound selected from:
(1-Benzyl-1H-imidazol-2-yl)methyl benzyl(phenyl)carbamate (Compound No. 1);
2-(2-Methyl-1H-imidazol-1-Dethyl benzyl(phenyl)carbamate (Compound No. 2);
2-(2-Methyl-1H-imidazol-1-yl)ethyl phenyl[4-(trifluoromethyl)benzyl]carbamate (Compound No. 3);
(1-Benzyl-1H-imidazol-2-yl)methyl phenyl [4-(trifluoromethyl)benzyl carbamate] (Compound No. 4);
2-(1H-imidazol-1-yl)ethyl benzyl(phenyl)carbamate (Compound No. 5);
(1-Benzyl-1,2,3,6-tetrahydropyridin-4-yl)methyl benzyl(phenyl)carbamate (Compound No. 6);
Pyridin-3-ylmethyl phenyl[4-(trifluoromethyl)benzyl]carbamate (Compound No. 7);
Pyridin-3-ylmethyl benzyl(phenyl)carbamate (Compound No. 8);
Pyridin-2-ylmethyl benzyl(phenyl)carbamate (Compound No. 9);
(1-Benzyl-1,2,5,6-tetrahydropyridin-3-yl)methyl benzyl(phenyl)carbamate (Compound No. 10);
Pyridin-4-ylmethyl benzyl(phenyl)carbamate (Compound No. 11);
2-(1H-Imidazol-1-yl)ethyl phenyl[4-(trifluoromethyl)benzyl]carbamate (Compound No. 12).

3. A pharmaceutical composition comprising a therapeutically effective amount of a compound as defined in claim 1 or 2 together with pharmaceutically acceptable carriers, excipients or diluents.

4. A method of treating or preventing disease or disorder of the respiratory, urinary or gastrointestinal systems, wherein the disease or disorder is mediated through muscarinic receptors, comprising administering to a patient in need thereof a therapeutically effective amount of a compound of claim 1.

5. A method of treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis comprising administering to a patient in need thereof a therapeutically effective amount of a compound of claim 1.

6. A pharmaceutical composition comprising one or more compounds of Formula I 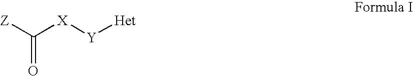
or a pharmaceutically accepted salts, pharmaceutically acceptable solvates, enantiomers, diastereomers, polymorphs or N-oxides thereof, wherein
Het is heterocyclyl or heteroaryl wherein nitrogen atom in heterocyclyl ring may also be quaternized to form a quaternary ammonium salts,
X is O, S or NR₁ (wherein R₁ is as defined below);
Y is no atom or —(CH₂)ₙ;
n is an integer from 1 to 6;
Z is —NHR₂, —N(R₂)₂ (wherein R₂ is as defined below), aryl or cycloalkyl;
R₁ is hydrogen, alkyl or aralkyl;
R₂ is independently selected from alkyl, aryl, aralkyl, heteroaryl, cycloalkyl, heterocyclyl, heterocyclylalkyl or heteroarylalkyl;

7. The method of preparing a compound of Formula VI and its pharmaceutically acceptable salts, pharmaceutically acceptable solvates, esters, enantiomers, diastereomers, N-oxides, polymorphs, prodrugs or metabolites,
wherein the reaction comprises of following steps:
a. reacting a compound of Formula II
(R₂)₂NH Formula II
with a compound of Formula III
hal-COOR_{z} Formula III
to form a compound of Formula IV, 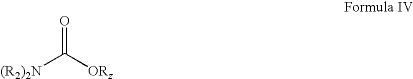
b. reacting a compound of Formula IV with a compound of Formula V
het-Y—OH Formula V
to give a compound of Formula VI 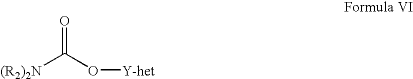
reducing a compound of Formula VI (when het is pyridyl) followed by reaction with a compound of Formula P-hal to give a compound of Formula VII 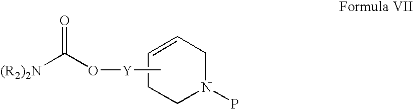
wherein
Het is heterocyclyl or heteroaryl wherein nitrogen atom in heterocyclyl ring may also be quaternized to form a quaternary ammonium salts,
Y is no atom or —(CH₂)ₙ;
R₂ is independently selected from alkyl, aryl, aralkyl, heteroaryl, cycloalkyl, heterocyclyl, heterocyclylalkyl or heteroarylalkyl;
R_{z} is alkyl or aryl;
hal Cl, Br or I;
P is selected from alkyl, cycloalkyl, heteroaryl, heterocyclyl, heteroarylalkyl, heterocyclylalkyl, aralkyl, —C(═O)OC(CH₃)₃, —C(═O)OC(CH₃)₂CHBr₂ or —C(═O)OC(CH₃)₂CCl₃;
